(19) United States
(12) Patent Application Publication
(10) Pub. No.: US 2005/0014960 A1
(43) Pub. Date: Jan. 20, 2005

(54) Catalyst preparation

(76) Inventors: Jan Karel Frederik Buijink, Amsterdam (NL); Mark Crocker, Lexington, KY (US); Carl Johan Gerrit Van Der Grift, Rodange (LU); Johannes Jacobus Maria Van Vlaanderen, Amsterdam (NL)

Correspondence Address: Jennifer D. Adamson;Shell Oil Company, Legal - Intellectual Property, P.O. Box 2463, Houston, TX, 77252-2463, US

(21) Appl. No.: 10/880,809

(22) Filed: Jun. 30, 2004

(30) Foreign Application Priority Data
Jun. 30, 2003 (EP) 03254162.5

(51) Int. Cl. B01J 27/135; C07D 301/06

(52) U.S. Cl. 549/533; 502/227

(57) ABSTRACT

The invention relates to a process for the preparation of an epoxidation catalyst which process involves: (a) drying a silica gel carrier at a temperature of from 400° C. to 1000° C.; (b) hydrolysing the dried silica gel carrier; (c) optionally drying the hydrolyzed carrier; and (d) contacting the carrier obtained with a gas stream containing titanium halide to obtain an impregnated carrier, in which process the hydrolysis of step (b) is carried out at a temperature of at most 200° C.

### FIELD OF THE INVENTION

The present invention relates to the preparation of an epoxidation catalyst and to the process of preparing alkylene oxide with the help of such catalyst.

### BACKGROUND OF THE INVENTION

An epoxidation catalyst is understood to be a catalyst which catalyzes the manufacture of an epoxy group containing compound. A known process comprises contacting organic hydroperoxide and alkene with a heterogeneous epoxidation catalyst and withdrawing a product stream comprising alkylene oxide and an alcohol.

Catalysts for the manufacture of an epoxy group containing compound are known. EP-A-345856 describes the preparation of such catalyst comprising impregnating a silicium compound with a stream of gaseous titanium tetrachloride. The example mentions that the silica was dried before the contact with titanium tetrachloride.

U.S. Pat. No. 6,114,552 teaches the use of a high surface area silica support in preparing epoxidation catalysts. The high surface area solid is impregnated with either a solution of a titanium halide in a non-oxygenated hydrocarbon solvent or a gas stream of titanium tetrachloride. It is mentioned that it is desirable to dry the silica support prior to impregnation, for example by heating for several hours at a temperature of at least 200 to 700° C. in order to attain a sufficient degree of dryness. The carrier is subsequently impregnated. In Example 1B, a silica support having a surface area of 1140 m²/g is heated to 400° C. and is then contacted with a stream of nitrogen and steam. Subsequently, the bed is cooled to 300° C.

There is a continuous interest in improving the performance of epoxidation catalysts in general, and more specifically of catalysts for the preparation of alkylene oxide.

WO 01/97967 specifically excludes silica gel carriers from its teaching. In Comparative Example 1, the commercial silicagel as such was loaded into the quartz reactor tube, heated to 260° C. under a nitrogen flow, cooled to 195° C. and subsequently contacted with gaseous tetrachloride. There is no information on how the silicagel was treated before it was loaded into the reactor tube. Someone skilled in the art will be aware that information on treatment of silica extrudate supports is not relevant for silica gel supports. Extrudates are very weak when coming out of the extruder and need special treatment for increasing their strength. One method for increasing the strength is calcination. Additionally, the water and extrusion aids present in extrudates can necessitate that the extrudates be subjected to further specific drying and/or calcining procedures.

### SUMMARY OF THE INVENTION

The present invention is directed to a process for the preparation of an epoxidation catalyst which process comprises:
(a) drying a silica gel carrier at a temperature of from 400° C. to 1000° C.;
(b) hydrolyzing the dried silica gel carrier;
(c) optionally drying the hydrolyzed carrier; and
(d) contacting the carrier obtained with a gas stream containing titanium halide to obtain an impregnated carrier,
   in which process the hydrolysis of step (b) is carried out at a temperature of at most 200° C.

### DETAILED DESCRIPTION OF THE INVENTION

Surprisingly, it was found that the performance of an epoxidation catalyst can be improved by a specific treatment before impregnation with a gaseous titanium halide. The improvement in performance was observed for each the conversion and the selectivity. In many cases, an improvement was observed in both the conversion and the selectivity.

The prior art contains no teaching or hint that the conversion and/or selectivity of an epoxidation catalyst can be improved by the combination of a specific drying step and a specific hydrolysis step.

The catalyst of the present invention is obtained by drying of a silica gel carrier followed by hydrolysis and impregnation with a titanium halide.

In principle, any silica gel carrier is suitable for use in the preparation process according to the present invention.

Contaminants may influence the performance of the final catalyst. It has been found that gas phase impregnation according to the present invention gives especially good results if the silica carrier contains at most 1200 ppm of sodium, more specifically at most 1000 ppm of sodium. Further, the silica carrier preferably comprises at most 500 ppm of aluminium, at most 500 ppm of calcium, at most 200 ppm of potassium, at most 100 ppm of magnesium and at most 100 ppm of iron.

The silica gel carrier for use in the present invention can in principle be any silica gel. Shaped extrudates of silica powder differ from silica gel carriers in their manufacturing method and in their physical properties. The high mechanical energy required to form the extrudate imparts high crushing strength and density to the extrudate but may decrease pore volume. A disadvantage of extrudates are the multiple steps required for obtaining extrudates of suitable strength. In general, silica gels are a solid, amorphous form of hydrous silicon dioxide distinguished from other hydrous silicon dioxides by their microporosity and hydroxylated surface. Silica gels usually contain three-dimensional networks of aggregated silica particles of colloidal dimensions. They are typically prepared by acidifying an aqueous sodium silicate solution, typically to a pH of less than 11, by combining it with a strong mineral acid. The acidification causes the formation of monosilicilic acid (Si(OH)₄), which polymerizes into particles with internal siloxane linkages and external silanol groups. The polymer particles aggregate, thereby forming chains and ultimately gel networks. Silicate concentration, temperature, pH and the addition of coagulants affect gelling time and final gel characteristics such as density, strength, hardness, surface area and pore volume. The resulting hydrogel is typically washed free of electrolytes, dried and activated. A suitable silica gel carrier would be silica support V432 and DAVICAT P-732, both of which are commercially available from Grace Davison.

The silica gel carrier for use in the present invention preferably has a surface area of at most 1000 m²/gram, more preferably at most 800 m²/gram, most preferably at most 500 m²/gram. Generally, the surface area will be at least 10 m²/gram, more specifically at least 20 m²/gram. Silica gel carriers which are found especially suitable have a surface area of 300 m²/g.

Preferably, silica gel carriers for use in the present invention have a weight average particle size of at most 2 millimetres. Silica gel carriers such as silica G 57 ex Grace, were found to be less preferred for use in the present invention. Particle sizes especially suitable for use in the present invention are weight average particle sizes of from 0.2 to 1.8 mm, more specifically of from 0.4 to 1.6 mm, most specifically of from 0.6 to 1.4 mm.

Drying according to the present invention comprises subjecting the silica gel carried to a temperature of from 400° C. to 1000° C. The temperature of the drying of step (a) is considered to be the temperature of the silica gel carrier. The drying can be carried out in the absence or in the presence of an inert gas such as nitrogen. Preferably, the drying is carried out at a temperature of from 450° C. to 900° C., more specifically at a temperature of from 500° C. to 850° C. The temperature chosen depends on the practical circumstances. Not all reactors can be used for subjecting the carrier to a relatively high temperature of about 850° C. However, such high temperature has been found to give especially good results.

The kind of silica gel used and the pretreatment of the silica gel influence the time which the drying is to be carried out. The drying will generally be carried out during from 15 minutes up to 10 hours, more specifically from 1 hour to 8 hours, more specifically of from 1 hour to 5 hours. The dried carrier obtained in step (a) is subsequently subjected to hydrolysis in step (b). The hydrolysis comprises treating the carrier with water and/or steam. The temperature of the hydrolysis of step (b) is considered to be the temperature of the catalyst while in contact with the water and/or steam. The temperature at which the hydrolysis is carried out is preferably from 10° C. to 200° C.

If the hydrolysis comprises treating the dried carrier with water, suitable methods for hydrolysis comprise pore impregnation treatment with water and soaking or immersing the dried carrier. Alternatively, the hydrolysis may comprise a washing treatment using water or an aqueous solution of a mineral acid, an aqueous solution of an ammonium salt or a combination thereof.

Any water which might still be present after the hydrolysis is preferably removed before treating the carrier further.

Preferably, the hydrolysis of step (b) comprises treating the carrier with steam. Steam which may be used is low pressure steam having a temperature of from 100° C. to 200° C., more specifically of from 120° C. to 180° C.

The desired temperature may be attained by a suitable combination of temperature of the carrier and temperature of the water and/or steam. It is preferred that the silica carrier has a temperature which is similar to the temperature of the water with which the carrier is treated.

It is preferred that a limited amount of water is added to the dried carrier, either in the form water or in the form of steam. Preferably, the amount of water is at most twice the pore volume of the carrier, more preferably at most 110% by volume. More preferably, the amount of water is at most 50% by volume. Most preferably, the amount of water is at most 40% by volume. The amounts of water are based on pore volume of the silica carrier. If steam is used, the amount of steam is taken as the volume which the same molar amount of water would have.

It was found that the silica gel carrier which had been treated in this way had the kind of surface which gave an excellent catalyst upon impregnation with gaseous titanium halide.

Preferably, the hydrolyzed carrier is dried. A suitable method for drying comprises contacting the hydrolyzed carrier with nitrogen at elevated temperature before contacting the gas stream containing titanium halide. The treatment is preferably carried out at a temperature of from 100° C. to 300° C., more specifically about 200° C. The duration of the treatment depends on the amount of water or steam added in step (b). Usually, the treatment will last from 0.5 hours to 2 hours.

Furthermore, it has been found especially advantageous if the amount of titanium halide supplied in step (d) is such that the molar ratio of titanium to silicon present in the carrier is from 0.050 to 0.063. It has been found that such molar ratio gives a more selective catalyst than similar catalysts of which the dried carrier had been in contact with either more titanium halide or less titanium halide. Without wishing to be bound to any theory, it is thought that this specific molar ratio gives a bonding of the titanium compounds which is especially advantageous for the selectivity of the catalyst.

Generally, the silica gel carrier is contacted with the titanium halide in the course of from 0.1 hour and 10 hours, more specifically of from 0.5 hours to 6 hours. Preferably, at least 30% wt of the titanium is added during the first 50% of the impregnation time. The time of impregnation is taken to be the time during which the silicon containing carrier is in contact with gaseous titanium halide. Most preferably, the silicon containing carrier is contacted with a similar amount of titanium halide during the full time of the impregnation. However, it will be clear to someone skilled in the art that deviations from this are allowable such as at the start of the impregnation, at the end of the impregnation and for relatively short time intervals during impregnation.

Titanium halides which may be used comprise tri- and tetra-substituted titanium complexes which have from 1 to 4 halide substituents with the remainder of the substituents, if any, being alkoxide or amino groups. The titanium halide may either be a single titanium halide compound or a mixture of titanium halide compounds. Preferably, the titanium halide comprises at least 50% wt of titanium tetrachloride, more specifically at least 70% wt of titanium tetrachloride. Most preferably, the titanium halide is titanium tetrachloride.

The present invention comprises the use of a gas stream comprising titanium halide. Preferably, the gas stream consists of titanium halide optionally in combination with an inert gas. If an inert gas is present, the inert gas preferably is nitrogen. Especially selective catalysts were found to be obtainable with the help of a gas stream solely consisting of titanium halide. In such process, the preparation is carried out in the absence of a carrier gas. However, limited amounts of further gaseous compounds are allowed to be present during the contact between the silicon containing carrier and the gaseous titanium halide. The gas in contact with the carrier during impregnation preferably consists for at least 70% wt of titanium halide, more specifically at least 80% wt, more specifically at least 90% wt, most specifically at least 95% wt. Specific preferred processes have been described in the co-pending patent application PCT/EP03/50875 claiming priority of European application No. 02252551.3.

Gaseous titanium halide may be prepared in any way known to someone skilled in the art. A simple and easy way comprises heating a vessel containing titanium halide to such temperature that gaseous titanium halide is obtained. If inert gas is to be present, the inert gas may be led over the heated titanium halide.

Generally, the impregnated carrier will be calcined and subsequently hydrolyzed and optionally silylated before being used as a catalyst. Therefore, the present invention further relates to a process further comprising (e) calcining the impregnated carrier obtained in step (d), (f) hydrolyzing the calcined impregnated carrier, and optionally (g) contacting the carrier obtained in step (f) with a silylating agent.

It is believed that calcination removes hydrogen halide, more specifically hydrogen chloride which is formed upon reaction of titanium halide and silicon compounds present on the surface of the silicon containing carrier.

The optional calcination of the impregnated carrier generally comprises subjecting the impregnated carrier to a temperature of at least 500° C., more specifically at least 600° C. Preferably, the calcination is carried out at a temperature of at least 650° C. From a practical point of view, it is preferred that the calcination temperature applied is at most 1000° C.

Hydrolysis of the impregnated and calcined carrier may remove titanium-halide bonds. The hydrolysis of the impregnated carrier generally will be somewhat more severe than the optional hydrolysis of the carrier before impregnation. Accordingly, this hydrolysis of the impregnated carrier is suitably carried out with steam at a temperature in the range of from 150° C. to 400° C.

Preferably, the hydrolyzed impregnated carrier is subsequently silylated. Silylation can be carried out by contacting the hydrolyzed impregnated carrier with a silylating agent, preferably at a temperature of between 100° C. and 425° C. Suitable silylating agents include organosilanes like tetra-substituted silanes with C₁-C₃ hydrocarbyl substituents. A very suitable silylating agent is hexamethyldisilazane. Examples of suitable silylating methods and silylating agents are, for instance, described in U.S. Pat. No. 3,829,392 and U.S. Pat. No. 3,923,843 which are referred to in U.S. Pat. No. 6,011,162, and in EP-A-734764, all of which are hereby incorporated by reference.

The amount of titanium (as metallic titanium) will normally be in the range of from 0.1% to 10% by weight, suitably 1% to 5% by weight, based on total weight of the catalyst. Preferably, titanium or a titanium compound, such as a salt or an oxide, is the only metal and/or metal compound present.

As mentioned above, it is known in the art to produce alkylene oxides, such as propylene oxide, by epoxidation of the corresponding olefin using a hydroperoxide such as hydrogen peroxide or an organic hydroperoxide as the source of oxygen. The hydroperoxide may be hydrogen peroxide or any organic hydroperoxide such as tert-butyl hydroperoxide, cumene hydroperoxide and ethylbenzene hydroperoxide. The alkene will generally be propene, which gives as alkylene oxide, propylene oxide. The catalyst prepared according to the present invention has been found to give especially good results in such process. Therefore, the present invention further relates to a process for the preparation of alkylene oxide which process comprises contacting a hydroperoxide and alkene with a heterogeneous epoxidation catalyst and withdrawing a product stream comprising alkylene oxide and an alcohol and/or water, in which process the catalyst is according to the present invention.

A specific organic hydroperoxide is ethylbenzene hydroperoxide, in which case the alcohol obtained is 1-phenylethanol. The 1-phenylethanol usually is converted further by dehydration to obtain styrene.

Another method for producing propylene oxide is the co-production of propylene oxide and methyl tert-butyl ether (MTBE) starting from isobutane and propene. This process is known in the art and involves similar reaction steps as the styrene/propylene oxide production process described in the previous paragraph. In the epoxidation step, tert-butyl hydroperoxide is reacted with propene forming propylene oxide and tert-butanol. Tert-butanol is subsequently etherified into MTBE.

A further method comprises the manufacture of propylene oxide from cumene. In this process, cumene is reacted with oxygen or air to form cumene hydroperoxide. Cumene hydroperoxide thus obtained is reacted with propene in the presence of an epoxidation catalyst to yield propylene oxide and 2-phenyl propanol. The latter may be converted into cumene a heterogeneous catalyst and hydrogen. Specific suitable processes are described for example in WO 02/48126, which is hereby incorporated by reference.

The conditions for the epoxidation reaction according to the present invention are those conventionally applied. For propene epoxidation reactions from ethylbenzene hydroperoxide, typical reaction conditions include temperatures of 50° C. to 140° C., suitably 75° C. to 125° C., and pressures up to 80 bar with the reaction medium being in the liquid phase.

The invention is further illustrated by the following Examples.

### EXAMPLES

The silica gel carrier used in the examples had a surface area of 300 m²/g, a pore volume of about 1.1 ml./g and a weight average particle size of about 1 mm. Substantially all particles had a particle size between 0.6 mm and 1.4 mm.

The silica gel carrier was dried at a temperature of 600° C. for 2 hours and was subsequently allowed to cool down. Different samples of the dried carrier were hydrolyzed in different ways.

Catalyst 1 was prepared by cooling the silica carrier to room temperature and subsequently impregnating the carrier with water. The amount of water was similar to the pore volume of the carrier. Excess water was subsequently removed by drying the carrier at 120° C. during 2 hours.

Catalyst 2 was prepared by cooling the silica carrier to a temperature of about 150° C. and contacting the carrier with steam having a temperature of 150° C.

Comparative catalyst 3 was prepared by cooling the silica carrier to a temperature of about 400° C. and contacting the carrier with steam having a temperature of 400° C.

The hydrolyzed carriers obtained were subsequently dried at about 250° C. in a nitrogen atmosphere for 2 hours and contacted with a gas stream consisting of titanium tetrachloride. The gas stream was obtained by heating titanium tetrachloride to 200° C. with the help of an electrical heating system. The silica carriers were impregnated such as to obtain impregnated carriers containing 3.6% wt of titanium on total amount of impregnated carrier.

The impregnated catalysts thus obtained were calcined at 600° C. during 7 hours. The calcined catalysts were subsequently contacted with steam at 325° C. for 6 hours. The steam flow consisted of 3 grams of water per hour and 8 Nl of nitrogen per hour. Finally, the catalysts were silylated at 185° C. for 2 hours by being contacted with 18 grams of hexamethyldisilazane per hour in a nitrogen flow of 1.4 Nl per hour.

The catalytic performance of the titanium catalyst samples was tested in an 1-octene batch test. In this test, 50 ml of a mixture containing 7.5% wt ethylbenzenehydroperoxide, 36% wt 1-octene and the remainder being ethylbenzene, was allowed to react in the presence of 1 g of catalyst at 40° C. while being mixed thoroughly. After 1 hour, the mixture was cooled in a mixture of ice and water to end the reaction. The concentrations of ethylbenzene hydroperoxide and 1-octene oxide are determined by (iodometric) titration.

In Table 1, the conversions and the selectivities are given for the catalysts derived from carriers hydrolyzed at different temperatures. The conversion is the percentage of ethylbenzenehydroperoxide which has been converted. The selectivity is the molar ratio of octene oxide formed to ethylbenzene hydroperoxide converted.

TABLE 1

| | Hydrolysis | | |
|---|---|---|---|
| | temperature | Conversion | Selectivity |
| | (° C.) | (%) | (%) |
| Catalyst 1 | ambient | 52.0 | 93.8 |
| Catalyst 2 | 150 | 48.7 | 93.1 |
| Comparative | 400 | 17.5 | 90.1 |
| catalyst 3 | | | |

1. A process for the preparation of an epoxidation catalyst which process comprises:
(a) drying a silica gel carrier at a temperature of from 400° C. to 1000° C.;
(b) hydrolyzing the dried silica gel carrier;
(c) optionally drying the hydrolyzed carrier; and,
(d) contacting the carrier obtained with a gas stream containing titanium halide to obtain an impregnated carrier,
in which process the hydrolysis of step (b) is carried out at a temperature of at most 200° C.

2. The process of claim 1, which process further comprises:
(e) calcining the impregnated carrier;
(f) hydrolyzing the calcined impregnated carrier; and, optionally
(g) contacting the carrier obtained in step (f) with a silylating agent.

3. The process of claim 2, wherein the hydrolysis of step (b) comprises treating the carrier with steam.

4. The process of claim 2, wherein the amount of titanium halide supplied in step (d) is such that the molar ratio of titanium halide added to silicon present in the carrier is from 0.050 to 0.063.

5. The process of claim 2, in which process the gas stream consists of titanium halide.

6. The process of claim 2, wherein the silica gel carrier has a surface area of at most 500 m²/g.

7. The process of claim 1, wherein the hydrolysis of step (b) comprises treating the carrier with steam.

8. The process of claim 1, wherein the amount of titanium halide supplied in step (d) is such that the molar ratio of titanium halide added to silicon present in the carrier is from 0.050 to 0.063.

9. The process of claim 1, in which process the gas stream consists of titanium halide.

10. The process of claim 1, wherein the silica gel carrier has a surface area of at most 500 m²/g.

11. A process for the preparation of alkylene oxide which process comprises contacting a hydroperoxide and alkene with a heterogeneous epoxidation catalyst and withdrawing a product stream comprising alkylene oxide and an alcohol and/or water, in which process the catalyst is prepared according to the process comprising:
(a) drying a silica gel carrier at a temperature of from 400° C. to 1000° C.;
(b) hydrolyzing the dried silica gel carrier;
(c) optionally drying the hydrolyzed carrier; and,
(d) contacting the carrier obtained with a gas stream containing titanium halide to obtain an impregnated carrier,
in which process the hydrolysis of step (b) is carried out at a temperature of at most 200° C.

12. The process of claim 11, in which process the alkene is propene and the alkylene oxide is propylene oxide.

13. The process of claim 11, wherein the hydroperoxide is ethylbenzene hydroperoxide and in which the alcohol is 1-phenylethanol.

14. The process of claim 13, which process further comprises dehydration of 1-phenylethanol to obtain styrene.
